(19) United States
(12) Patent Application Publication
(10) Pub. No.: US 2004/0120457 A1
(43) Pub. Date: Jun. 24, 2004

(54) Scatter reducing device for imaging

(75) Inventors: Andrew Karellas, Atlanta, GA (US); Sankararaman Suryanarayanan, Atlanta, GA (US); Srinivasan Vedantham, Atlanta, GA (US)

Correspondence Address: THOMAS O. HOOVER, ESQ., BOWDITCH & DEWEY, LLP, 161 Worcester Road, P.O. Box 9320, Framingham, MA, 01701-9320, US

(73) Assignee: University of Massachusetts Medical Center, Worcester, MA (US)

(21) Appl. No.: 10/327,639

(22) Filed: Dec. 20, 2002

(51) Int. Cl. A61B 6/14

(52) U.S. Cl. 378/62

(57) ABSTRACT

The present invention related to a system and method for performing scatter correction in x-ray imaging systems. A pixellated solid state imaging detector is used in which an electronic window or slot is scanned across the two dimensional surface of the detector to selectively record image data. In a preferred embodiment, a collimator is used to define relative movement between an x-ray beam and the x-ray detector. A scatter correction program can be used to correct for scattering in the detected image data to provide for improved imaging in medical, scientific and industrial applications.

### CROSS REFERENCES TO RELATED APPLICATIONS

The present application claims the benefit of U.S. Provisional Application No. 60/344,306, filed Dec. 21, 2001. The entire contents of the above application is incorporated herein by reference in its entirety.

### GOVERNMENT SUPPORT

This invention was supported, in whole or in part, by a grant R01 CA88792 from National Institutes For Health. The Government has certain rights in the invention.

### BACKGROUND OF THE INVENTION

The most common method of x-ray scatter reduction is the antiscatter grid. It is a device with a series of lead blades or lamellae lined up in parallel that preferentially absorb scattered radiation and passes primarily non-scattered radiation. A variety of x-ray scatter reduction approaches using linear grids, crossed grids, focused grids, parallel grids, moving grids, and air gaps have been studied. X-ray antiscatter grids are used in x-ray imaging, and they are important for imaging the adult chest, abdomen and pelvis by conventional radiography. The main problem with antiscatter grids is their inability to provide complete “clean-up” of scattered radiation. For example, eliminating 70% of the scattered radiation may be attainable but achieving a 95% reduction in scattered radiation is extremely difficult and impractical with conventional or even with special purpose antiscatter grids. Antiscatter grids with high scatter rejection capability also absorb primary radiation. Absorption of primary radiation must be compensated with higher radiation dose to the patient in order to maintain a constant signal-to-noise ratio on the image detector. Therefore the high rejection of scattered radiation with the use of a grid is associated with higher radiation dose to the patient.

Another system uses an x-ray tube collimated to irradiate only the linear detector array with a fan beam thereby preventing unnecessary exposure of other areas. Both the tube and detector move in synchrony over the region to be exposed. A problem with this scanning procedure is the time required to perform the scan, typically four to ten seconds, which for some examinations such as chest imaging, is considered slow, and patient motion can affect spatial resolution to some degree. Another system utilizes an image intensifier and thresholds that are compared on a pixel by pixel basis. This system is bulky and requires extensive computation on a frame-by-frame basis. There is a continuing need, however, for improvements in image quality, reducing x-ray exposure, smaller footprint and the cost of manufacture of such instruments, particularly for medical imaging applications.

### SUMMARY OF THE INVENTION

The present invention relates to the removal of x-ray scatter in imaging applications such as mammography, bone densitometry of the spine, the hip, the hand and other peripheral joints, chest radiography and other medical, scientific, and industrial applications. In a preferred embodiment, the invention can be added to existing flat panel imaging systems for scatter reduction with minimal modifications. Scattered x-rays can reduce image contrast which can adversely affect the diagnostic quality of images in medical applications, for example. Moreover, scattered radiation can severely affect the normally linear relationship between exposure and signal on an electronic imaging detector thereby rendering the image useless for quantitative studies such as bone densitometry. The present invention uses a scanning electronic slot without the need for image intensifiers or processing signals outside of the slot. The electronic slot can be used with a scanning mechanical slot that is controlled to match the scanning movement of the electronic slot.

A preferred embodiment of the invention uses a scanning collimator that is positioned between the x-ray source and the x-ray detector to define and control an x-ray beam that is scanned across an object to be imaged and the detector surface. A preferred embodiment uses an area detector that is stationary relative to the object or patient and a scanning electronic slot or window. The shape, size and movement of the electronic slot or active region of the detector are correlated with the same parameters for the slot assembly such that the beam transmitted through the mechanical slot or window without being scattered is aligned with the electronic slot or window of the detector. As the area of the detector receiving the beam at any give time is known, scattered x-rays that are received by the detector outside the reception area do not contribute to the detected image data. A programmable computer with associated software or a dedicated processor can be used to control scanning parameters and provide the needed data processing. The individual recorded windows from each frame can be assembled to form a complete image from the scanned region using a software module.

The detector can be any pixellated solid state detector such as a charge coupled device (CCD), a CMOS imager or amorphous silicon sensor or an x-ray sensitive detectors such as amorphous selenium that convert x-rays directly into electrical signals. Individual pixels can be controlled by colocated thin film transistors, for example, that allows the user to select regions of pixel elements to define the reception window at any given moment. A preferred embodiment of the invention utilizes a pre-exposure scan in which the amount of scatter is measured for the region of interest. Depending on the thickness and size of the region of interest, the x-ray source parameters, the collimator area and scan parameters and electronic slot parameters are selected, and the scan is performed. The size of the collimator slot and the electronic slot can be adjusted manually or automatically depending upon the pre-exposure step. Unlike previously described systems, no thresholding is necessary, but it can be used with this system for certain applications. Pixel elements are selectively actuated to perform readout. In another preferred embodiment of the invention, the electronic slot width and other parameters can be set automatically or manually without pre-exposure.

A plurality of apertures or slots in the collimator can be used to provide for lower scan times. A preferred embodiment employs a plurality of parallel fan beams that are scanned simultaneously across the detector surface. Additionally an x-ray monitor can be used to detect and record the quality of the beams. The monitoring system can provide automatic calibration or shut-off of the system if a selected deviation occurs in the detected signal. The detector can utilize binning of adjoining pixel elements to improve scan time and signal to noise ratio.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A illustrates a scatter reducing system in accordance with a preferred embodiment of the invention.

FIG. 1B schematic illustrating embodiments employing more than one scanning slot.

FIG. 2A shows a detector system for slot scan showing a single slot scanning a flat-panel detector array.

FIG. 2B illustrates an image processing system using “image stitching” to generate a fully reconstructed image.

FIG. 3 illustrates an interleaved scanning process where the slot (or multiple slots) scans the detector area in either a discrete or continuous fashion.

FIG. 4 illustrates an x-ray quality monitoring device-used to assess x-ray beam quality exiting from the tube.

FIGS. 5A and 5B are process sequences illustrating methods of performing a scanning sequence in accordance with the invention.

### DETAILED DESCRIPTION OF THE INVENTION

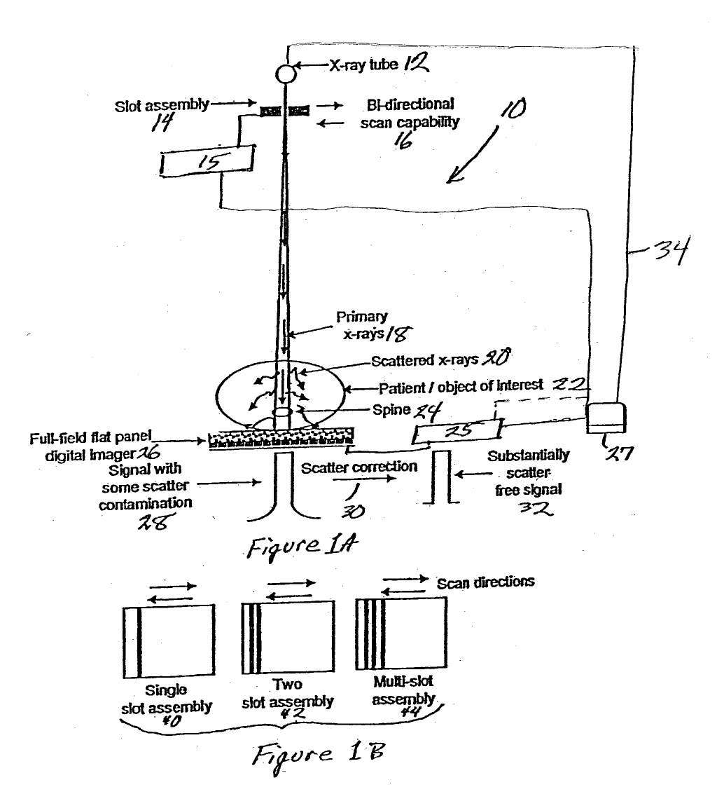

A schematic of a preferred embodiment of the invention is illustrated in FIG. 1A. The system differs from the existing slot-scan technology in that an aperture of the collimator “steers” the x-ray beam that scans a flat-panel or wide area detector 26. Only the area illuminated by the slot assembly 14 allows x-rays 18, from source 12 to pass through while the rest of the area blocks x-rays, preventing unnecessary exposure to the patient or other object of interest 22. The slot width, height, and scan speed of a single slot 40 can be adjusted either manually or automatically as dictated by the diagnostic task. Multiple slot assemblies 42, 44 can also be used depending on the scattering 20 requirements of the application (FIG. 1B).

An electronic interface 25 can be used to selectively read-out the information under the slot area. Image data can be stored in memory, processed and displayed using a data processing system or personal computer 27. The control system and actuator 15 can be used with a bi-directional scanning capability 16. The computer 27 can be connected to and programmed to control the system 15 as well as the readout function of interface 25. Alternatively, post-acquisition image processing techniques can be used to reconstruct the image by adding or “stitching” different image frames. An important advantage of the invention is the lack of post-patient x-ray beam collimating. Post-patient beam collimating is traditionally considered essential to protect the detector 26 from contamination with scattered radiation. However, the present electronic slot scanning system and multi-frame electronic acquisition technique described herein provides for imaging without mechanical post-patient collimating. This is important because of the use of a post-patient slit greatly complicates the device. It requires synchronization, it is prone to produce artifacts, and it adds cost and bulk to the equipment.

Digital imaging systems can be easily adapted to perform quantitative studies such as bone densitometry. Moreover, with this method the user can perform bone densitometry with scanning at two distinct energy levels with hitherto unattainable spatial detail. Although high spatial detail is not critical for all bone densitometry, there are situations where physicians can use higher spatial resolution. Most importantly, conversion to the slot scan is simple as it involves minimal hardware modifications, and the rest is done by electronic control of the digital detector.

The present invention can be used for other x-ray imaging applications such as mammography. Digital mammographic systems can perform in the slot scan mode (typically within a small selected area) as an option to the conventional single snapshot acquisition. The slot scan approach in mammography can be especially useful in dense breasts where x-ray scatter interferes with visualization of subtle contrast. The detector system can comprise a cassette assembly having a size and shape suitable for replacing a standard film cassette.

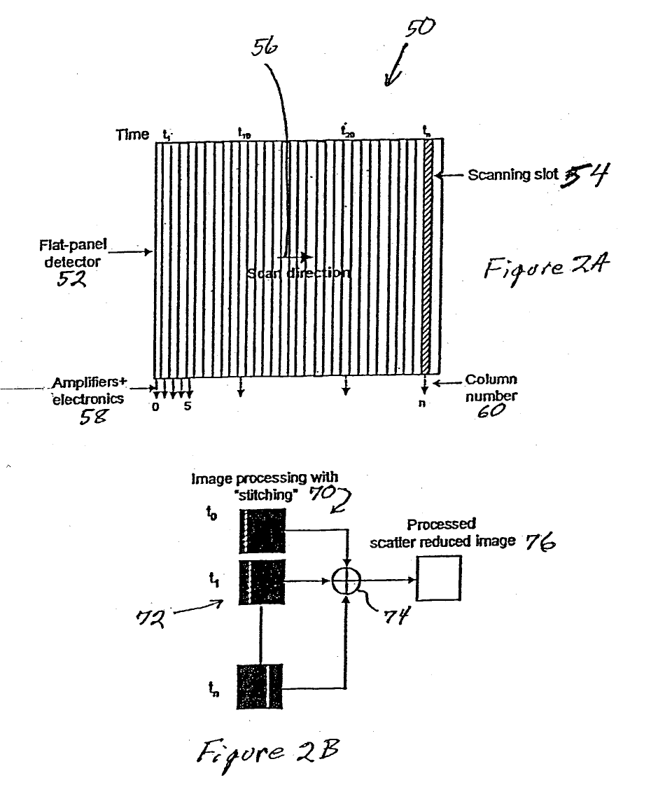

In this embodiment 50 (FIG. 2A), a read-out circuitry 58 can be used to selectively read the data under the slot 54 as it is traversing 56 a predefined area of detector 52. This acts as an “electronic window” that reads the data directly under the slot that can correspond to a particular column 60 while eliminating unwanted information. A substantially “scatter free” 76 image can then be displayed after completion of the scan (FIG. 2B).

A complete scanned image can be obtained in almost “real time” using this procedure and the systems can be less expensive compared to traditional slot-scan units. In this embodiment the existing flat-panel detectors and electronics can be used and multiple images (frames) can be acquired as the slot is scanned. The area under the slot can be selectively extracted from each frame 72. Filtering can be used to remove unwanted components. The extracted portions can then be added or “stitched” 70 using image-processing techniques to generate a full resolution “scatter free” image (FIG. 2B). This method is portable and requires the addition of only the software module to control the slot assembly, detector readout and processing.

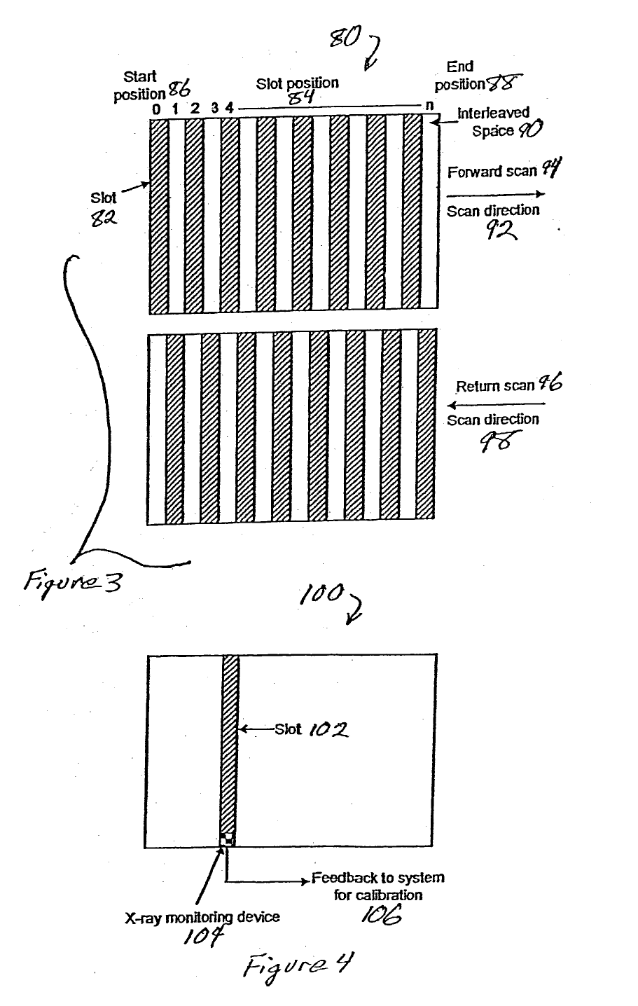

Different types of scanning modes can be used for task specific imaging applications. For a continuous scan, for example, the slot scans the detector area in a continuous fashion. The direction of the scan can be changed when required. X-rays are ‘ON’ during the scan. For a discrete scan, the slot 82 scans 92 the detector area in uniform discrete steps. Here, the x-rays need to be ‘ON’ only when the slot has arrived at a specific position and remain ‘OFF’ during the transition. For an interleaved scan, the slot interleaves columns 90 when scanning the detector either in discrete steps or continuously. For example, columns 0, 2, 4 . . . n (assume ‘n’ is even) are scanned in the forward scan and columns n−1, . . . , 3, 1 are scanned when the slot returns to its start position (column 0) (FIG. 3). Further, the scans can be performed either from left to right as a forward scan 94, a return scan 96 or top to bottom with reference to the patient or object of interest.

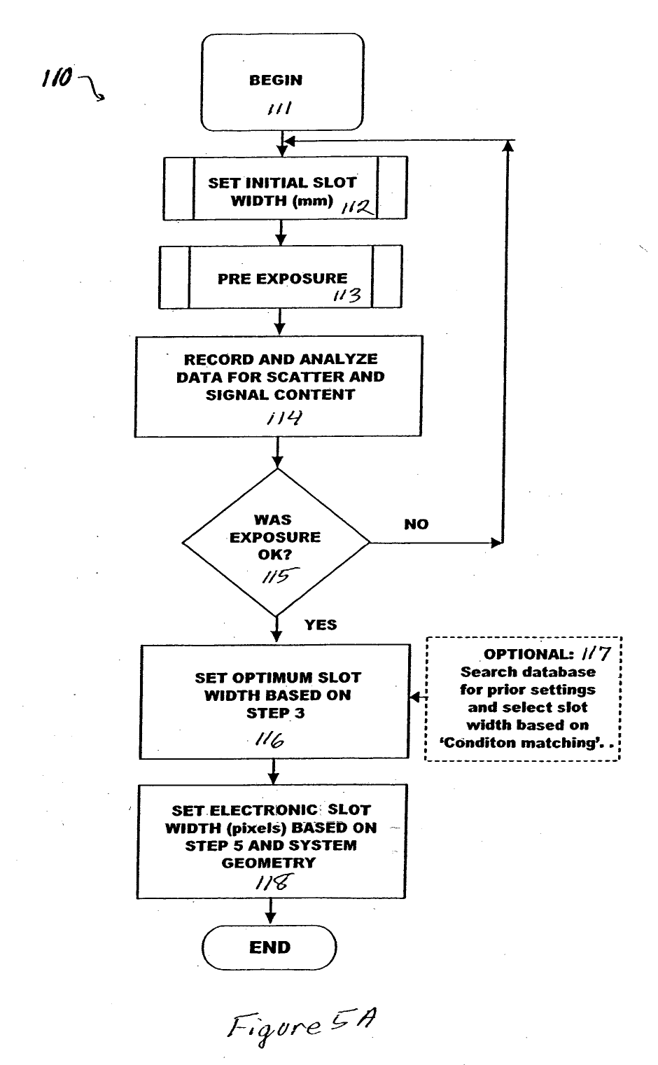

In certain applications, such as bone densitometry, it is useful to monitor 100 the exit beam quality from the tube. In the present invention certain standard x-ray attenuating materials, such as aluminum, bone, or other appropriate material on or adjacent to the slot 102 (FIG. 4) and recording the intensity of the signal under the material. If the signal deviates over a predefined amount, the system controller or computer 27 is triggered by feedback signal 106 to calibrate the source 12 via connection 34 or stop under extreme circumstances. Alternatively, a small section of the flat panel detector can be used for the beam monitoring function.

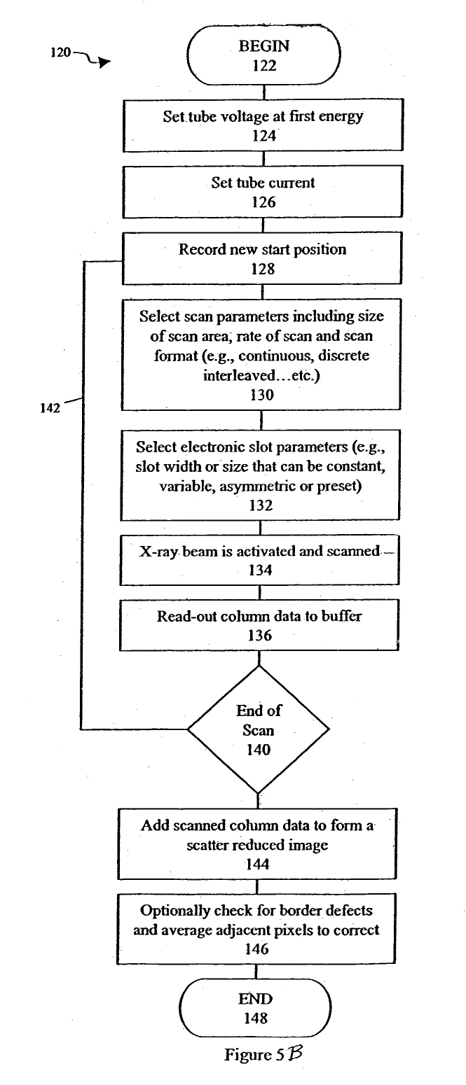

During a typical bone density scan, the system operates, as shown in FIGS. 5A and 5B. The operator activates the system which can include a pre-exposure sequence to measure scatter and thereby assist in setting actual scan parameters. In the pre-exposure sequence of FIG. 5A, the user initiates 111 the scan by setting an initial slot size 112. The pre-exposure 113 is performed, the data is recorded and analyzed 114. If the scan is not acceptable the scan can be rerun or the actual scan can then be programmed, 116 and 118. In a preferred embodiment a database can be referenced 117 to check or refine parameters. The actual scan in FIG. 5B shows the tube voltage set 124 at one energy, such as 60kV, and an appropriate filter such as aluminum is automatically inserted in the beam; the tube current is set 126 to a relatively low value, typically 5 to 20 mA; the starting position is selected and recorded 128, scan parameters are selected 130, including size of scan area, rate of scan and scan format (e.g., a continuous scan, a discrete scan of selected regions, or an interleaved scan). These parameters can be set automatically on the object thickness and composition. In the case of medical imaging, this can include patient data and the portion of the anatomy to be scanned. Next, electronic slot parameters (e.g. slot width or size that can be constant, variable, asymmetric or preset at a selected value) are selected 132.

The x-ray beam is activated, and it is scanned 134 across the detector while the electronic readout is synchronized with the beam scan as described in the above modes; this image is read out 136 and stored in the computer as the “low energy” image; the scan is repeated 142 or replaced or a higher x-ray beam energy (for example 100 kV) is selected with another filter, typically aluminum or copper, or a combination of each, which can be automatically inserted in the beam; this image is acquired in the same manner of the first (low energy image); and the second image is stored in the computer as the “high energy” image. Prior to storage of each image, the columns can simply be added 144, and in the event of border defects the operator can optionally select 146 to check for border defects and select adjacent pixel values to be averaged to correct those defects. The data can then be processed to determine the bone density of the region of interest from the low and high energy images.

Portability of the scanning unit and compatibility with any existing wide area digital imager (scintillator with amorphous silicon readout, amorphous selenium with amorphous silicon readout, cadmium zinc telluride, crystalline silicon, scintillator with active or passive type-CMOS readout, scintillator with charge-coupled device detector and readout, phosphor detectors and other monolithically fabricated integrated detector devices). Additional details regarding x-ray sources, detectors and methods of scanning and processing image data can be found in U.S. Pat. Nos. 5,150,394 and 6,031,892, incorporated herein by reference in their entirety.

Additional embodiments employ variable slots (which may be adaptive) for task specific applications; the use of hardware data read-out; and the use of software image processing (including “image stitching”). Adaptive scanning can be performed using the feedback control system or can be programmed for specific applications or patients.

Another preferred embodiment comprises a dedicated bone densitometer. A flat-panel-based bone densitometer provides more cost efficiency than the current generation of bone densitometers. Dual energy bone densitometers can be used to make quantitative measurements of the spinal to measure bone loss, for example. Moreover, it delivers much higher performance and has fewer moving parts than the current generation of such devices that are based on mechanical scanning of the entire x-ray tube and detector.

While this invention has been particularly shown and described with references to preferred embodiments thereof, it will be understood by those skilled in the art that various changes in form and details may be made therein without departing from the scope of the invention encompassed by the appended claims.

What is claimed:

1. An x-ray imaging system comprising:
an x-ray source emitting an x-ray beam; and
a solid state x-ray imaging detector having a two dimensional array of pixel elements, the pixel elements being selectively actuated to provide a scanning window that scans across the detector.

2. The imaging system of claim 1 further comprising a scanning collimator positioned between the source and the detector and an actuator that controls movement of the scanning collimator.

3. The imaging system of claim 1 further comprising a controller connected to the actuator and the imaging detector that controls movement of the collimator and an electronic window on the detector.

4. The imaging system of claim 1 wherein the collimator further comprises a plurality of apertures that define a plurality of beams scanning a surface of the detector.

5. The imaging system of claim 1 further comprising a data processor connected to the detector, the data processor performing scatter correction of image data.

6. The imaging system of claim 1 wherein the solid state imaging detector comprises a scintillator and a charge coupled device.

7. The imaging system of claim 1 wherein the solid state imaging detector comprises a CMOS imaging device.

8. The imaging system of claim 1 wherein the solid state imaging detector comprises a monolithic, pixellated flat panel device.

9. The imaging system of claim 1 further comprising a control circuit connected to the x-ray source that detects and adjusts x-ray intensity.

10. The imaging system of claim 1 further comprising a data processor that assembles an image from a plurality of image frames.

11. The imaging system of claim 2 wherein a size of a window in the collimator can be adjusted during a scan.

12. The imaging system of claim 2 wherein the collimator has a plurality of slots.

13. The imaging system of claim 1 further comprising a data processor programmed to perform a pre-exposure scan.

14. The imaging system of claim 3 wherein the window comprises an adjustable array of pixel elements of an amorphous silicon sensor.

15. A method of processing an image comprising:
providing an x-ray source and detector; and
detecting the x-ray beam with a detector having a scanning window that scans to form an electronic representation of an object to be imaged.

16. The method of claim 15 further comprising actuating a scanning movement of an electronic slot on the detector.

17. The method of claim 15 further comprising the detector with a plurality of slots.

18. The method of claim 15 further comprising performing a bone density measurement.

19. The method of claim 15 further comprising the steps of selecting window scan parameters and slot parameters of a scanning slot positioned between the source and detector.

20. The method of claim 15 further comprising interleaving slots during a scan.

21. The method of claim 15 further comprising performing a pre-exposure scan to select scan parameters.

22. The method of claim 15 further comprising controlling a beam characteristic during the scan.

23. The method of claim 15 further comprising performing a mammographic scan.

24. The method of claim 15 further comprising detecting the beam with a scintillator and a silicon detector.

25. The method of claim 15 further comprising detecting without a scintillator or an image intensifier.

26. A method of making a scanning slit x-ray system comprising:
providing an x-ray source and a solid state imaging detector; and
connecting a programmable computer to the detector and a scanning slot, the computer being programmed to provide an electronic window on the detector that scans with the slot across an object to be imaged.

27. The method of claim 26 further comprising an electronic controller connected to the detector and the computer.

28. The method of claim 26 further comprising programming the computer to assemble an image of the object from a plurality of frames.

29. The method of claim 26 further comprising controlling a slot size and a window size to reduce scatter.

30. The method of claim 26 providing a dual energy x-ray source.

31. A bone densitometer comprising:
an x-ray source emitting an x-ray beam;
a solid state x-ray imaging detector having a two dimensional array of pixel elements; and
a scanning collimator positioned between the source and the detector, the scanning collimator controlling direction of the x-ray beam such that the beam scans across the detector.

32. The imaging system of claim 31 further comprising an actuator that controls movement of the scanning collimator.

33. The imaging system of claim 31 further comprising a controller connected to the actuator and the imaging detector that controls movement of the collimator and an electronic window on the detector.

34. The imaging system of claim 31 wherein the collimator further comprises a plurality of apertures that define a plurality of beams scanning a surface of the detector.

35. The imaging system of claim 31 further comprising a data processor connected to the detector, the data processor performing scatter correction of image data.

36. A method of forming an image comprising:
providing an x-ray source and detector;
actuating relative movement between a collimator and the detector to scan a beam of x-rays across a detector surface; and
detecting the x-ray beam at a first energy and a second energy with the detector.

37. The method of claim 36 further comprising actuating a scanning movement of an electronic slot on the detector.

38. The method of claim 36 further comprising the detector with a plurality of slots.

39. The method of claim 36 further comprising performing a bone density measurement.

40. The method of claim 36 further comprising the steps of inputting patient data into a computer to select scan parameters and slot parameters.

41. The imaging system of claim 1 wherein the detector comprises amorphous selenium.

42. The imaging system of claim 1 wherein the detector directly converts x-rays into electrical signals.

43. The imaging system of claim 1 further comprising a feedback control system to monitor and adjust the x-ray beam.

44. The imaging system of claim 3 wherein the controller addresses individual pixel elements to control readout of image data.

45. The imaging system of claim 3 wherein the controller actuates a continuous scan, a discrete step scan, an interleaved scan or a binned scan.
